(19) United States
(12) Patent Application Publication
(10) Pub. No.: US 2012/0165285 A1
(43) Pub. Date: Jun. 28, 2012

(54) COMBINED PREPARATION FOR USE AS A MEDICAMENT

(76) Inventor: Peter Richardson, Hadstock (GB)

(21) Appl. No.: 13/382,885

(22) PCT Filed: Jul. 9, 2010
(86) PCT No.: PCT/GB2010/001324

§ 371 Date: Mar. 19, 2012

(30) Foreign Application Priority Data
Jul. 9, 2009 (GB) 0911982.7
Nov. 16, 2009 (GB) 0920045.2

(51) Int. Cl. A61K 31/7076; A61P 29/00; A61P 35/00; A61P 9/00; A61P 9/10; A61P 25/08; A61P 25/28; A61P 3/10; A61P 25/00; A61P 37/00

(52) U.S. Cl. 514/46

(57) ABSTRACT

A combined preparation comprising an A_{2A} adenosine receptor agonist and a calcium channel blocker is described. The effect of the A_{2A} adenosine receptor agonist is enhanced in the presence of the calcium channel blocker. Methods for treatment of pathological conditions using the combined preparation are described.

This invention relates to a combined preparation for co-administration or sequential administration to a subject, and to methods for treating pathological conditions, in particular pain or inflammation, using the combined preparation.

Adenosine is a ubiquitous local hormone/neurotransmitter that acts on four known receptors, the A₁, A_{2A}, A_{2B} and A₃ adenosine receptors. Agonism of A_{2A} adenosine receptors is known to have analgesic and anti-inflammatory effects.

It has now surprisingly been found that the effect of an A_{2A} adenosine receptor agonist is enhanced in the presence of a calcium channel blocker.

According to the invention there is provided a combined preparation comprising an A_{2A} adenosine receptor agonist and a calcium channel blocker.

Preferably the preparation is for co-administration or sequential administration of the A_{2A} adenosine receptor agonist and the calcium channel blocker to the subject, more preferably a human subject. For co-administration, the A_{2A} adenosine receptor agonist and the calcium channel blocker may be provided as a mixture, or they may be separate from each other to allow simultaneous administration.

Preferably the combined preparation includes a pharmaceutically acceptable carrier, excipient, or diluent. If the A_{2A} adenosine receptor agonist and the calcium channel blocker are separate from each other in the preparation, they may each be together with a pharmaceutically acceptable carrier, excipient, or diluent, which may be the same, or a different pharmaceutically acceptable carrier, excipient, or diluent.

There is also provided according to the invention a combined preparation of the invention for use as a medicament.

There is also provided according to the invention a combined preparation of the invention for preventing, treating, or ameliorating a pathological condition that can be prevented, treated, or ameliorated by agonism of an A_{2A} adenosine receptor.

There is also provided according to the invention use of a combined preparation of the invention in the manufacture of a medicament for preventing, treating, or ameliorating a pathological condition that can be prevented, treated, or ameliorated by agonism of an A_{2A} adenosine receptor.

Examples of pathological conditions that can be prevented, treated, or ameliorated by agonism of an A_{2A} adenosine receptor are pain, inflammation, cancer, auto-immune disease, ischemia-reperfusion injury, epilepsy, sepsis, septic shock, neurodegeneration, or vascular complications of diabetes, in particular microvascular complications of diabetes, including diabetic neuropathy, diabetic neuropathic pain, diabetic skin ulceration and dermopathy, diabetic kidney disease, or diabetic retinopathy, or macrovascular complications of diabetes, including cardiovascular disease (including atherosclerosis and claudication associated with cardiovascular disease), and heart disease (including atherosclerosis associated with heart disease).

A_{2A} adenosine receptor agonists are well known to the skilled person. Examples are described in U.S. Pat. No. 5,877,180, WO 2003/086408. It has previously been found that spongosine (2-methoxyadenosine), is an effective analgesic at doses as much as one hundred times lower than would be expected to be required based on the known affinity of this compound for adenosine receptors. At these doses, spongosine does not cause the significant side effects associated with higher doses of this compound, or other adenosine receptor agonists. Thus, the therapeutic effects of spongosine can be separated from its side effects. The activity of spongosine as an analgesic is the subject of International patent application no. PCT/GB03/05379, and the activity of compounds related to spongosine as analgesics is the subject of International patent application no. PCT/GB04/00935. Use of spongosine and related compounds to treat inflammation and other disorders is the subject of International patent application no. PCT/GB04/000952:

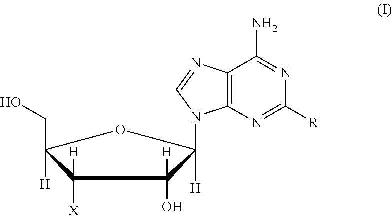

wherein R is C₁₋₄ alkoxy and X is OH or H.

It was previously reported that spongosine, and the related compounds described in PCT/GB04/00935 and PCT/GB04/000952, have increased affinity for adenosine receptors at pH below pH 7.4. It is believed that this property explains the surprising activity of these compounds at low doses. The Applicant has been able to identify certain other compounds that also have increased affinity for adenosine receptors at reduced pH. It is thought that these compounds can be used as medicaments without causing serious side effects. These compounds are described in PCT/GB2005/000800, and are covered by the following formulae:

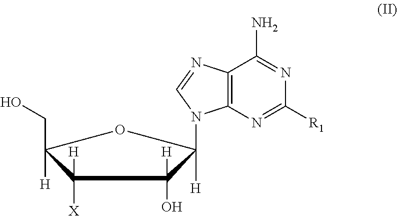

wherein:
when X═OH, R₁ is C₁ or C₄-C₆ alkoxy (preferably C₅-C₆ alkoxy), OCH₂Cyclopropyl, OCH₂Cyclopentyl, O-(2,2,3,3-tetrafluoro-cycloButyl), phenoxy, substituted phenoxy (preferably substituted with nitrile (preferably 4-nitrile), 4-methyl, phenyl (preferably 3-phenyl), 3-bromo, 3-isopropyl, 2-methyl, 2,4-difluoro, 2,5-difluoro, 3,4-difluoro, 2,3,5-trifluoro, or (3-methyl, 4-fluoro)), OCH₂CH₂OH, OCH₂CHF₂, (5-indanyl)oxy, C₁, C₂, C₅, or C₆ alkylamino, (R) or (S)-sec-Butylamino, C₅ or C₆ cycloalkylamino, exo-norbornane amino, (N-methyl, N-isoamylamino), phenylamino, phenylamino with either methoxy or fluoro substituents, a C₂ sulfone group, a C₇ alkyl group, a cyano group, a CONH₂ group, or 3,5-dimethylphenyl; or
when X═H, R₁ is n-hexyloxy;

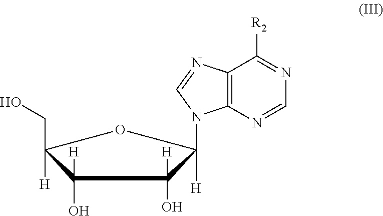

wherein R₂ is NMe₂, N-(2-isopentenyl), piperazinyl, (N-Me, N-benzyl), (N-Me, N—CH₂Ph(3-Br)), (N-Me, N—CH₂Ph(3-CF₃)), or (N-Me, N-(2-methoxyethyl)), or OCH₂Cyclopentyl;

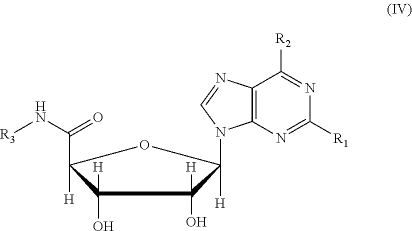

wherein:
when R₁═H, R₃ is an isopropyl group, and R₂ is either NH₂, a methylamino group (NHMe) or an isoamyl group (CH₂CH₂CHMe₂); or
when R₁═H, R₃ is H, and R₂ is NH₂; or
when R₁ is OMe, R₃ is Ph, and R₂ is NH₂; or
when R₁ is NHCH₂CH₂CH₂CH₂CH₂Me, R₃ is CH₂CH₂CH₂Me, and R₂ is NH₂;

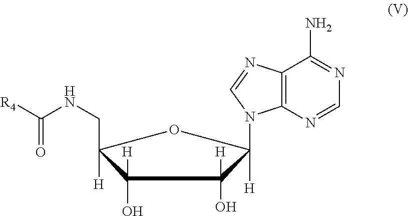

wherein R₄ is n-propyl or NHCH₂CH₃;

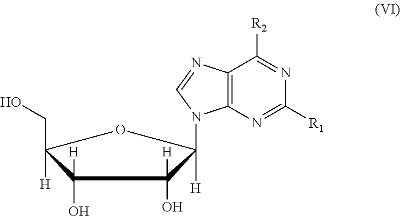

wherein:
R₁ is NHCyclohexyl when R₂ is NMe₂; or
R₁ is OMe when R₂ is NHBenzyl;

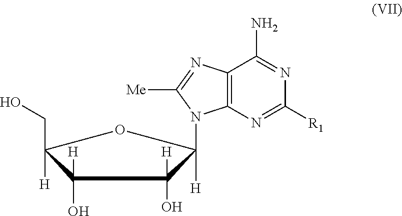

wherein R1 is NHCyclohexyl, NHCyclopentyl, or NH-n-Hexyl;
or a pharmaceutically acceptable salt thereof.

The term “alkyl” is used herein to mean an unsubstituted straight or branched chain hydrocarbon group. Preferably the alkyl is straight chain.

The term “alkoxy” is used herein to mean an unsubstituted straight or branched chain alkyl-oxy group. Preferably the alkoxy is a straight chain alkyl-oxy group.

The term “C₁, C₂, C₅, or C₆ alkylamino” is used herein to mean a group —NR^{x}R^{y} in which R^{x} is hydrogen and R^{y} is C₁, C₂, C₅, or C₆ alkyl, or in which R^{x} and R^{y} are each independently C₁, C₂, C₅, or Cₛ alkyl. Preferably R^{x} and R^{y} are each C₁ alkyl.

Compounds of Formulae (I)-(VII) are all believed to have increased affinity for adenosine receptors at pH below pH 7.4. In normal mammalian tissues plasma pH is tightly regulated between pH 7.35 and 7.45. Some tissues experience lower pH values, particularly the lumen of the stomach (pH between 2 and 3) and the surfaces of some epithelia (for example, the lung surface pH is approximately 6.8). In pathological tissues, for example during inflammation, ischaemia and other types of damage, a reduction in pH occurs.

Because of the increased affinity of the compounds of Formulae (I)-(VII) for adenosine receptors at reduced pH, it is thought that the actions of these compounds can be targeted to regions of low pH, such as pathological tissues. Consequently, the doses of these compounds that are required to give therapeutic effects are much lower than would be expected based on their affinity for adenosine receptors at normal extracellular physiological pH. Since only low doses of the compounds are required, the serious side effects associated with administration of adenosine receptor agonists are avoided or minimised. This has the surprising consequence (contrary to the teaching in the art, for example in U.S. Pat. No. 5,877,180) that some adenosine receptor agonists that are low affinity and/or non-selective agonists at physiological pH (such as spongosine) can be therapeutically effective without causing serious side effects.

In view of the enhanced effect of an A2a adenosine receptor agonist in the presence of a calcium channel blocker, it is believed that A2a adenosine receptor agonists that do not have increased affinity for adenosine receptors pH below pH 7.4 may be administered at lower doses than would otherwise be required, thereby reducing the side effects of such conventional A2a adenosine receptor agonists.

It is preferred, however, that the A_{2A} adenosine receptor agonist is an A_{2A} adenosine receptor agonist of any of the above Formulae (I)-(VII) or a pharmaceutically acceptable salt thereof. Particularly preferred A_{2A} adenosine receptor agonists are compounds of formula (I), most preferably spongosine (also known as 2-methoxyadenosine, 9H-purin-6-amine, 9-α-D-arabinofuranosyl-2-methoxy).

Examples of pharmaceutically acceptable salts are organic addition salts formed with acids which form a physiologically acceptable anion, for example, tosylate, methanesulphonate, malate, acetate, citrate, malonate, tartarate, succinate, benzoate, ascorbate, α-ketoglutarate, and α-glycerophosphate. Suitable inorganic salts may also be formed, including hydrochloride, sulphate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium, or lithium) or alkaline earth metal (for example calcium) salts of carboxylic acids can also be made.

Calcium channel blockers are conventionally used to decrease blood pressure in individuals with hypertension. Calcium channel blockers work by blocking voltage-gated calcium channels (VGCCs) in cardiac muscle and blood vessels. This decreases intracellular calcium leading to a reduction in muscle contraction. In the heart, a decrease in calcium available for each beat results in a decrease in cardiac contractility. In blood vessels, a decrease in calcium results in less contraction of the vascular smooth muscle and therefore an increase in arterial diameter (CCB's do not work on venous smooth muscle), a phenomenon called vasodilation. Vasodilation decreases total peripheral resistance, while a decrease in cardiac contractility decreases cardiac output. Since blood pressure is determined by cardiac output and peripheral resistance, blood pressure drops.

Several types of blockers of L-type voltage-gated calcium channels are well known to the skilled person, and include dihydropyridines, phenylalkylamines, and benzothiazepines.

Dihydropyridine calcium channel blockers are often used to reduce systemic vascular resistance and arterial pressure, but are not used to treat angina (with the exception of amlodipine and nifedipine, which carry an indication to treat chronic stable angina as well as vasospastic angina) because the vasodilation and hypotension can lead to reflex tachycardia. Examples include Amlodipine (Norvasc, Azor), Aranidipine (Sapresta), Azelnidipine (Calblock), Barnidipine (HypoCa), Benidipine (Coniel), Cilnidipine (Atelec, Cinalong, Siscard), Clevidipine (Cleviprex), Efonidipine (Landel), Felodipine (Plendil), Lacidipine (Motens, Lacipil), Lercanidipine (Zanidip), Manidipine (Calslot, Madipine), Nicardipine (Cardene, Carden SR), Nifedipine (Procardia, Adalat), Nilvadipine (Nivadil), Nimodipine (Nimotop), Nisoldipine (Baymycard, Sular, Syscor), Nitrendipine (Cardif, Nitrepin, Baylotensin), Pranidipine (Acalas).

Phenylalkylamine calcium channel blockers are relatively selective for myocardium, reduce myocardial oxygen demand and reverse coronary vasospasm, and are often used to treat angina. They have minimal vasodilatory effects compared with dihydropyridines. Their action is intracellular. Examples include Verapamil (Calan, Isoptin), Gallopamil (Procorum, D600).

Benzothiazepine calcium channel blockers are an intermediate class between phenylalkylamine and dihydropyridines in their selectivity for vascular calcium channels. By having both cardiac depressant and vasodilator actions, benzothiazepines are able to reduce arterial pressure without producing the same degree of reflex cardiac stimulation caused by dihydropyridines. An example is Diltiazem (Cardizem).

While most of the calcium channel blockers listed above are relatively selective, there are also agents that are considered nonselective. These include mibefradil, bepridil, fluspirilene, and fendiline.

Any of the above calcium channel blockers are suitable for use in the present invention.

There is also provided according to the invention a method of prevention, treatment, or amelioration of a pathological condition that can be prevented, treated, or ameliorated by agonism of an A_{2A} adenosine receptor, which comprises administering an A_{2A} adenosine receptor agonist and a calcium channel blocker to a subject in need of such prevention, treatment, or amelioration.

In particular, administration of an A₂, adenosine receptor agonist and a calcium channel blocker in accordance with the invention may be used for the prevention, treatment, or amelioration of pain, cancer, inflammation, auto-immune disease, ischemia-reperfusion injury, epilepsy, sepsis, septic shock, neurodegeneration (including Alzheimer's Disease), muscle fatigue, muscle cramp, and vascular complications of diabetes, in particular microvascular complications of diabetes, including diabetic neuropathy, diabetic neuropathic pain, diabetic skin ulceration and dermopathy, diabetic kidney disease, or diabetic retinopathy, or macrovascular complications of diabetes, including cardiovascular disease, (including atherosclerosis and claudication associated with cardiovascular disease), and heart disease (including atherosclerosis associated with heart disease).

Certain aspects of the invention relate to the treatment of pain. Pain has two components, each involving activation of sensory neurons. The first component is the early or immediate phase when a sensory neuron is stimulated, for instance as the result of heat or pressure on the skin. The second component is the consequence of an increased sensitivity of the sensory mechanisms innervating tissue which has been previously damaged. This second component is referred to as hyperlagesia, and is involved in all forms of chronic pain arising from tissue damage, but not in the early or immediate phase of pain perception.

Thus, hyperalgesia is a condition of heightened pain perception caused by tissue damage. This condition is a natural response of the nervous system apparently designed to encourage protection of the damaged tissue by an injured individual, to give time for tissue repair to occur. There are two known underlying causes of this condition, an increase in sensory neuron activity, and a change in neuronal processing of nociceptive information which occurs in the spinal cord. Hyperalgesia can be debilitating in conditions of chronic inflammation (e.g. rheumatoid arthritis), and when sensory nerve damage has occurred (i.e. neuropathic pain).

The preparations and methods of the invention may be used for the prevention, treatment, or amelioration of pain (particularly hyperalgesia) caused as a result of neuropathy, including Diabetic Neuropathy, Polyneuropathy, Cancer Pain, Fibromyalgia, Myofascial Pain Syndrome, Osteoarthritis, Pancreatic Pain, Pelvic/Perineal pain, Post Herpetic Neuralgia, Rheumatoid Arthritis, Sciatica/Lumbar Radiculopathy, Spinal Stenosis, Temporo-mandibular Joint Disorder, HIV pain, Trigeminal Neuralgia, Chronic Neuropathic Pain, Lower Back Pain, Failed Back Surgery pain, back pain, post-operative pain, post physical trauma pain (including gunshot, road traffic accident, burns), Cardiac pain, Chest pain, Pelvic pain/PID, Joint pain (tendonitis, bursitis, acute arthritis), Neck Pain, Bowel Pain, Phantom Limb Pain, Obstetric Pain (labour/C-Section), Renal Colic, Acute Herpes Zoster Pain, Acute Pancreatitis Breakthrough Pain (Cancer), Dysmenorhoea/Endometriosis.

The preparations and methods of the invention may be used for the prevention, treatment, or amelioration of pain (particularly hyperalgesia) caused as a result of the microvascular complications of diabetes, including diabetic neuropathy, diabetic neuropathic pain, diabetic skin ulceration and dermopathy, diabetic kidney disease, or diabetic retinopathy.

The preparations and methods of the invention may be used for the prevention, treatment, or amelioration of pain (particularly hyperalgesia) caused as a result of inflammatory disease, or as a result of combined inflammatory, autoimmune and neuropathic tissue damage, including rheumatoid arthritis, osteoarthritis, rheumatoid spondylitis, gouty arthritis, and other arthritic conditions, cancer, HIV, chronic pulmonary inflammatory disease, silicosis, pulmonary sarcosis, bone resorption diseases, reperfusion injury (including damage caused to organs as a consequence of reperfusion following ischaemic episodes e.g. myocardial infarcts, strokes), autoimmune damage (including multiple sclerosis, Guillam Barre Syndrome, myasthenia gravis) graft v. host rejection, allograft rejections, fever and myalgia due to infection, AIDS related complex (ARC), keloid formation, scar tissue formation, Crohn's disease, ulcerative colitis and pyresis, irritable bowel syndrome, osteoporosis, cerebral malaria and bacterial meningitis, bowel pain, cancer pain, back pain, fibromyalgia, post-operative pain, bladder cystitis.

The preparations and methods of the invention may be used for the prevention, treatment, or amelioration of ischaemic pain. The term “ischaemic pain” is used herein to mean pain associated with a reduction in blood supply to a part of the body. A reduced blood supply limits the supply of oxygen (hypoxia) and energy to that part of the body. Ischaemia arises from poor blood perfusion of tissues and so ischaemic pain arises in coronary artery disease, peripheral artery disease, and conditions which are characterized by insufficient blood flow, usually secondary to atherosclerosis. Other vascular disorders can also result in ischaemic pain. These include: left ventricular hypertrophy, coronary artery disease, essential hypertension, acute hypertensive emergency, cardiomyopathy, heart insufficiency, exercise tolerance, chronic heart failure, arrhythmia, cardiac dysrhythmia, syncopy, arteriosclerosis, mild chronic heart failure, angina pectoris, Prinzmetal's (variant) angina, stable angina, and exercise induced angina, cardiac bypass reocclusion, intermittent claudication (arteriosclerosis oblitterens), arteritis, diastolic dysfunction and systolic dysfunction, atherosclerosis, post ischaemia/reperfusion injury, diabetes (both Types I and II), thromboembolisms. Haemorrhagic accidents can also result in ischaemic pain. In addition poor perfusion can result in neuropathic and inflammatory pain arising from hypoxia-induced nerve cell damage (e.g. in cardiac arrest or bypass operation, diabetes or neonatal distress).

The preparations and methods of the invention may be used for the prevention, treatment, or amelioration of inflammation. In particular, inflammation caused by or associated with: cancer (such as leukemias, lymphomas, carcinomas, colon cancer, breast cancer, lung cancer, pancreatic cancer, hepatocellular carcinoma, kidney cancer, melanoma, hepatic, lung, breast, and prostate metastases, etc.); auto-immune disease (such as organ transplant rejection, lupus erythematosus, graft v. host rejection, allograft rejections, multiple sclerosis, rheumatoid arthritis, type I diabetes mellitus including the destruction of pancreatic islets leading to diabetes and the inflammatory consequences of diabetes); retinopathy; nephropathy; neuropathy; diabetes, in particular the vascular complications of diabetes, including the microvascular complications of diabetes, and the macrovascular complications of diabetes, skin disorder; autoimmune damage (including multiple sclerosis, Guillain Barre Syndrome, myasthenia gravis); obesity; cardiovascular conditions associated with poor tissue perfusion and inflammation (such as atheromas, atherosclerosis, stroke, ischaemia-reperfusion injury, claudication, spinal cord injury, congestive heart failure, vasculitis, haemorrhagic shock, vasospasm following subarachnoid haemorrhage, vasospasm following cerebrovascular accident, pleuritis, pericarditis, the cardiovascular complications of diabetes); ischaemia-reperfusion injury, ischaemia and associated inflammation, restenosis following angioplasty and inflammatory aneurysms; epilepsy, neurodegeneration (including Alzheimer's Disease), muscle fatigue or muscle cramp (particularly athletes' cramp), arthritis (such as rheumatoid arthritis, osteoarthritis, rheumatoid spondylitis, gouty arthritis), fibrosis (for example of the lung, skin and liver), multiple sclerosis, sepsis, septic shock, encephalitis, infectious arthritis, Jarisch-Herxheimer reaction, shingles, toxic shock, cerebral malaria, Lyme's disease, endotoxic shock, gram negative shock, haemorrhagic shock, hepatitis (arising both from tissue damage or viral infection), deep vein thrombosis, gout; conditions associated with breathing difficulties (e.g. chronic obstructive pulmonary disease, impeded and obstructed airways, bronchoconstriction, pulmonary vasoconstriction, impeded respiration, chronic pulmonary inflammatory disease, silicosis, pulmonary sarcosis, cystic fibrosis, pulmonary hypertension, pulmonary vasoconstriction, emphysema, bronchial allergy and/or inflammation, asthma, hay fever, rhinitis, vernal conjunctivitis and adult respiratory distress syndrome); conditions associated with inflammation of the skin (including psoriasis, eczema, ulcers, contact dermatitis); conditions associated with inflammation of the bowel (including Crohn's disease, ulcerative colitis and pyresis, irritable bowel syndrome, inflammatory bowel disease); HIV (particularly HIV infection), cerebral malaria, bacterial meningitis, TNF-enhanced HIV replication, TNF inhibition of AZT and DDI activity, osteoporosis and other bone resorption diseases, osteoarthritis, rheumatoid arthritis, infertility from endometriosis, fever and myalgia due to infection, cachexia secondary to cancer, cachexia secondary to infection or malignancy, cachexia secondary to acquired immune deficiency syndrome (AIDS), AIDS related complex (ARC), keloid formation, scar tissue formation, adverse effects from amphotericin B treatment, adverse effects from interleukin-2 treatment, adverse effects from OKT3 treatment, or adverse effects from GM-CSF treatment, and other conditions mediated by excessive anti-inflammatory cell (including neutrophil, eosinophil, macrophage and T-cell) activity.

Continuous low grade inflammation is known to be associated with obesity (in the presence and absence of insulin resistance and Type II diabetes) (Browning et al (2004) Metabolism 53, 899-903, Inflammatory markers elevated in blood of obese women; Mangge et al (2004) Exp Clin Endocrinol Diabetes 112, 378-382, Juvenile obesity correlates with serum inflammatory marker C-reactive protein; Maachi et al Int J Obes Relat Metab Disord. 2004 28, 993-997, Systemic low grade inflammation in obese people). A possible reason for this is that fat cells secrete TNF alpha and interleukins 1 and 6, which are pro-inflammatory.

The preparations and methods of the invention may be used for the prevention, treatment, or amelioration of vascular complications of diabetes, in particular microvascular complications of diabetes, including diabetic neuropathy, diabetic neuropathic pain, diabetic skin ulceration and dermopathy, diabetic kidney disease, or diabetic retinopathy, or microvascular complications of diabetes, including cardiovascular disease, or heart disease. Without being bound by theory, it is believed that agonism of adenosine A_{2A} receptors is able to treat the microvascular complications of diabetes by causing dilatation, as well as by treating the associated inflammation. It is also believed that agonism of adenosine A_{2A} receptors is able to treat the macrovasular complications of diabetes, in particular cardiovascular disease (including atherosclerosis and claudication associated with cardiovascular disease) by treating the associated inflammation and atheroma formation, and heart disease (including atherosclerosis associated with heart disease) by causing dilatation, treating the associated inflammation, and inhibiting ischaemia reperfusion injury.

A_{2A} adenosine receptor agonists that are selective A_{2A} adenosine receptor agonists are particularly preferred because it is believed that such compounds will have strong anti-inflammatory activity. By selective A_{2A} adenosine receptor agonists is meant agonists that activate A_{2A} adenosine receptors at concentrations that are lower (preferably one thousandth to one fifth) than required to activate A₁ adenosine receptors. Furthermore, A₁ adenosine receptors have pro-inflammatory activity, so such effects are expected to be minimised for compounds that are selective for A_{2A} adenosine receptors.

A person of ordinary skill in the art can readily test whether or not a pathological condition that is prevented, treated, or ameliorated by a compound of formula (I)-(VII) is acting via A_{2A} adenosine receptors. For example, this may be done by comparing the effect of the compound in an animal model of the pathological condition in the presence and absence of a selective antagonist of an A_{2A} adenosine receptor. If the effect of the compound in the presence of the antagonist is reduced or absent compared with the effect of the compound in the absence of the antagonist, it is concluded that the compound is exerting its effect via an A_{2A} adenosine receptor. Antagonists of A_{2A} adenosine receptors are known to those of ordinary skill in the art (see for example Ongini et al., Farmaco. 2001 January-February; 56(1-2):87-90; Muller, Curr Top Med. Chem. 2003; 3(4):445-62).

Alternatively, an A_{2A} adenosine receptor knockout mouse may be used (Ohta A and Sitkovsky M, Nature 2001; 414:916-20). For example, the effect of the compound on a mouse that has symptoms of the pathological condition is compared with its effect on an A_{2A} adenosine receptor knockout mouse that has corresponding symptoms. If the compound is only effective in the mouse that has A_{2A} adenosine receptors it is concluded that the compound is exerting its effect via A_{2A} adenosine receptors.

The A_{2A} adenosine receptor agonist and the calcium channel blocker may be co-administered to the subject, either simultaneously or as a mixture, or they may be administered sequentially to the subject.

The A_{2A} adenosine receptor agonist and the calcium channel blocker may be packaged together (for example as a mixture) for co-administration, or in the same packaging but separately from one another for co-administration or sequential administration. The A_{2A} adenosine receptor agonist and the calcium channel blocker may be packaged with a set of instructions for administration of the combined preparation. Where the A_{2A} adenosine receptor agonist and the calcium channel blocker are packaged together for co-administration, a set of instructions may be provided for co-administration. Alternatively, where the A_{2A} adenosine receptor agonist and the calcium channel blocker are packaged separately from one another for co-administration or sequential administration, a set of instructions may be provided for co-administration or sequential administration.

For sequential administration, the A_{2A} adenosine receptor agonist may be administered to the subject before or after administration of the calcium channel blocker. It will be appreciated that the A_{2A} adenosine receptor agonist should still be in active form in the subject when the calcium channel blocker is administered, and vice versa. Preferably the A_{2A} adenosine receptor agonist is administered to the subject after administration of the calcium channel blocker.

The A_{2A} adenosine receptor agonist and the calcium channel blocker may be administered to the subject within minutes of each other or within 48 hours of each other. Preferably the A_{2A} adenosine receptor agonist and the calcium channel blocker are administered to the subject within 24 hours, preferably within 12 hours, of each other.

The A_{2A} adenosine receptor agonist and the calcium channel blocker can conveniently be administered in a pharmaceutical preparation containing the A_{2A} adenosine receptor agonist and the calcium channel blocker in combination with a pharmaceutically acceptable carrier, excipient, or diluent. If the A_{2A} adenosine receptor agonist and the calcium channel blocker are separate from each other in the preparation, they may each be combined with a pharmaceutically acceptable carrier, excipient, or diluent, which may be the same, or a different pharmaceutically acceptable carrier, excipient, or diluents.

Pharmaceutical compositions can be prepared by methods and contain carriers, excipients, or diluents which are well known in the art. A generally recognized compendium of such methods and ingredients is Remington's Pharmaceutical Sciences by E. W. Martin (Mark Publ. Co., 15th Ed., 1975). The compounds and compositions of the present invention can be administered parenterally (for example, by intravenous, intraperitoneal or intramuscular injection), topically, orally, or rectally.

For oral therapeutic administration, the active compounds may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavouring agent such as peppermint, oil of wintergreen, or cherry flavouring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compounds of the preparation may be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

Pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions can be prepared by incorporating the active compounds in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the active compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the A_{2A} adenosine receptor agonist and the calcium channel blocker can be determined by comparing their in vitro activity, and in vivo activity in animal models. Methods for the extrapolation of effective dosages in mice, and other animals, to humans are known to the art; for example, see U.S. Pat. No. 4,938,949.

The A_{2A} adenosine receptor agonist and the calcium channel blocker are conveniently administered in unit dosage form; for example, containing about 0.05 mg to about 500 mg, conveniently about 0.1 mg to about 250 mg, most conveniently, about 1 mg to about 150 mg of active ingredient per unit dosage form. The A_{2A} adenosine receptor agonist and the calcium channel blocker may be combined in a single unit dose, or the AZ_{A} adenosine receptor agonist and calcium channel blocker may be provided each as separate unit dose.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The active compounds can conveniently be administered orally, sublingually, transdermally, or parenterally at dose levels of about 0.01 to about 150 μg/kg, preferably about 0.1 to about 50 μg/kg, and more preferably about 0.1 to about 10 μg/kg of mammal body weight.

For parenteral administration the compounds are presented in aqueous solution in a concentration of from about 0.1 to about 10%, more preferably about 0.1 to about 7%. The solution may contain other ingredients, such as emulsifiers, antioxidants or buffers.

The amount of a compound of formula (I)-(VII) (or other A_{2A} adenosine receptor agonist that has increased affinity for A_{2A} adenosine receptor at pH below pH 7.4) that is administered to a subject is preferably an amount which gives rise to a peak plasma concentration that is less than the EC50 value of the compound at A_{2A} adenosine receptors (preferably at pH 7.4).

Preferably the peak plasma concentration of the compound is one ten thousandth to one half (or one ten thousandth to one fifth, or one ten thousandth to one twentieth, or one ten thousandth to one hundredth, or one ten thousandth to one thousandth, or one thousandth to one half, or one thousandth to one fifth, or one thousandth to one twentieth, or one fiftieth to one tenth, or one hundredth to one half, or one hundredth to one fifth, or one fiftieth to one third, or one fiftieth to one half, or one fiftieth to one fifth, or one tenth to one half, or one tenth to one fifth) of the EC50 value.

Preferably the amount of a compound that is administered gives rise to a plasma concentration that is maintained for more than one hour at one ten thousandth to one half (or one ten thousandth to one fifth, or one ten thousandth to one twentieth, or one ten thousandth to one hundredth, or one ten thousandth to one thousandth, or one thousandth to one half, or one thousandth to one fifth, or one thousandth to one twentieth, or one fiftieth to one tenth, or one hundredth to one half, or one hundredth to one fifth, or one fiftieth to one half, or one fiftieth to one fifth, or one tenth to one half, or one tenth to one fifth) of the EC50 value of the compound at A_{2A} adenosine receptors.

Preferably the amount administered gives rise to a plasma concentration that is maintained for more than one hour between one thousandth and one half, or one thousandth and one fifth, or one thousandth and one twentieth, or one hundredth and one half, or one hundredth and one fifth, or one fiftieth and one half, or one fiftieth and one fifth, of the EC50 value of the compound at A_{2A} adenosine receptors at pH 7.4.

For the avoidance of doubt, the EC50 value of a compound is defined herein as the concentration of the compound that provokes a receptor response halfway between the baseline receptor response and the maximum receptor response (as determined, for example, using a dose-response curve).

The EC50 value should be determined under standard conditions (balanced salt solutions buffered to pH 7.4). For EC50 determinations using isolated membranes, cells and tissues this would be in buffered salt solution at pH 7.4 (e.g. cell culture medium), for example as in Daly et al., Pharmacol. (1993) 46, 91-100), or preferably as in Tilburg et al (J. Med. Chem. (2002) 45, 91-100). The EC50 could also be determined in vivo by measuring A_{2A} adenosine receptor mediated responses in a normal healthy animal, or even in a tissue perfused under normal conditions (i.e. oxygenated blood, or oxygenated isotonic media, also buffered at pH 7.4) in a normal healthy animal.

Alternatively, the amount of the compound that is administered may be an amount that results in a peak plasma concentration that is less than the Kd value of the compound at A_{2A} adenosine receptors. Preferably the peak plasma concentration of the compound is one ten thousandth to one half (or one ten thousandth to one fifth, or one ten thousandth to one twentieth, or one ten thousandth to one hundredth, or one ten thousandth to one thousandth, or one thousandth to one half, or one thousandth to one third, or one thousandth to one fifth, or one thousandth to one twentieth, or one fiftieth to one tenth, or one hundredth to one half, or one hundredth to one fifth, or one fiftieth to one half, or one fiftieth to one fifth, or one tenth to one half, or one tenth to one fifth) of the Kd value.

Preferably the amount of the compound that is administered is an amount that results in a plasma concentration that is maintained for at least one hour between one thousandth and one half, or one thousandth and one fifth, more preferably between one thousandth and one twentieth, or one hundredth and one half, or one hundredth and one fifth, or one fiftieth and one half, or one fiftieth and one fifth, of the Kd value of the compound at A_{2A} adenosine receptors.

Preferably the amount of the compound that is administered is an amount that results in a plasma concentration that is maintained for more than one hour at one ten thousandth to one half (or one ten thousandth to one fifth, or one ten thousandth to one twentieth, or one ten thousandth to one hundredth, or one ten thousandth to one thousandth, or one thousandth to one half, or one thousandth to one fifth, or one thousandth to one twentieth, or one fiftieth to one tenth, or one hundredth to one half, or one hundredth to one fifth, or one fiftieth to one half, or one fiftieth to one fifth, or one fiftieth to one third, or one tenth to one half, or one tenth to one fifth) of the Kd value of the compound at A_{2A} adenosine receptors.

The Kd value of the compound at each receptor should be determined under standard conditions using plasma membranes as a source of the A_{2A} adenosine receptors derived either from tissues or cells endogenously expressing these receptors or from cells transfected with DNA vectors encoding the adenosine receptor genes. Alternatively whole cell preparations using cells expressing A_{2A} adenosine receptors can be used. Labelled ligands (e.g, radiolabelled) selective for the different receptors should be used in buffered (pH 7.4) salt solutions (see e.g. Tilburg et al, J. Med. Chem. (2002) 45, 420-429) to determine the binding affinity and thus the Kd of the compound at A_{2A} adenosine receptors.

Alternatively, the amount of the compound that is administered may be an amount that is one ten thousandth to one half (or one ten thousandth to one fifth, or one ten thousandth to one twentieth, or one ten thousandth to one hundredth, or one ten thousandth to one thousandth, or one thousandth to one half, or one thousandth to one fifth, or one thousandth to one twentieth, or one fiftieth to one tenth, or one hundredth to one half, or one hundredth to one fifth, or one fiftieth to one half, or one fiftieth to one third, or one fiftieth to one fifth, or one tenth to one half, or one tenth to one fifth) of the minimum amount (or dose) of the compound that gives rise to bradycardia, hypotension or tachycardia side effects in animals of the same species as the subject to which the compound is to be administered. Preferably the amount administered gives rise to a plasma concentration that is maintained for more than one hour at one ten thousandth to one half (or one ten thousandth to one fifth, or one ten thousandth to one twentieth, or one ten thousandth to one hundredth, or one ten thousandth to one thousandth, or one thousandth to one half, or one thousandth to one fifth, or one thousandth to one twentieth, or one fiftieth to one tenth, or one hundredth to one half, or one hundredth to one fifth, or one fiftieth to one half, or one fiftieth to one fifth, or one tenth to one half, or one tenth to one fifth) of the minimum amount of the compound that gives rise to the side effects.

Preferably the amount administered gives rise to a plasma concentration that is maintained for more than 1 hour between one thousandth and one half, or one thousandth and one twentieth, or one hundredth or one fiftieth and one half, or one hundredth or one fiftieth and one fifth of the minimum dose that gives rise to the side effects.

Alternatively, the amount of the compound that is administered may be an amount that gives rise to plasma concentrations that are one ten thousandth to one half (or one ten thousandth to one fifth, or one ten thousandth to one twentieth, or one ten thousandth to one hundredth, or one ten thousandth to one thousandth, or one thousandth to one half, or one thousandth to one fifth, or one thousandth to one twentieth, or one fiftieth to one tenth, or one hundredth to one half, or one hundredth to one fifth, or one fiftieth to one half, or one fiftieth to one third, or one fiftieth to one fifth, or one tenth to one half, or one tenth to one fifth) of the minimum plasma concentration of the compound that cause bradycardia, hypotension or tachycardia side effects in animals of the same species as the subject to which the compound is to be administered. Preferably the amount administered gives rise to a plasma concentration that is maintained for more than one hour at one ten thousandth to one half (or one ten thousandth to one fifth, or one ten thousandth to one twentieth, or one ten thousandth to one hundredth, or one ten thousandth to one thousandth, or one thousandth to one half, or one thousandth to one fifth, or one thousandth to one twentieth, or one fiftieth to one tenth, or one hundredth to one half, or one hundredth to one fifth, or one fiftieth to one half, or one fiftieth to one fifth, or one tenth to one half, or one tenth to one fifth) of the minimum plasma concentration of the compound that causes the side effects.

Preferably the amount administered gives rise to a plasma concentration that is maintained for more than 1 hour between one thousandth and one half, or one thousandth and one twentieth, or one hundredth or one fiftieth and one half, or one hundredth or one fiftieth and one fifth, of the minimum plasma concentration that causes the side effects.

The appropriate dosage of the compound will vary with the age, sex, weight, and condition of the subject being treated, the potency of the compound (such as its EC50 value for an A_{2A} adenosine receptor), its half life, its absorption by the body, and the route of administration, etc. However, the appropriate dosage can readily be determined by one skilled in the art.

A suitable way to determine the appropriate dosage is to assess cardiovascular changes (for example by ecg and blood pressure monitoring) at or around the EC50 value of the compound for an A_{2A} adenosine receptor to determine the maximum tolerated dose. The therapeutically effective dose is then expected to be one ten thousandth to one half (or one ten thousandth to one fifth, or one ten thousandth to one twentieth, or one ten thousandth to one hundredth, or one ten thousandth to one thousandth, or one thousandth to one half, or one thousandth to one fifth, or one thousandth to one twentieth, or one fiftieth to one tenth, or one hundredth to one half, or one hundredth to one fifth, or one fiftieth to one half, or one fiftieth to one third, or one fiftieth to one fifth, or one tenth to one half, or one tenth to one fifth) of the maximum tolerated dose.

For spongosine, the dose should be less than 28 mg in humans. This dose gives rise to plasma concentrations between 0.5 and 0.9 μM (close to the Kd at adenosine A2A receptors at pH 7.4 see below). Based on this result, the preferred dosage range for spongosine is 0.03 to 0.3 mg/kg.

The minimum plasma concentration of spongosine giving maximal analgesic relief in a rat adjuvant model of arthritis was 0.06 μM, considerably less than the EC50 of spongosine at the adenosine A2A receptor which is approximately 1 μM. The preferred dosing levels in humans give maximum plasma concentrations between 0.005 and 0.5 μM which are significantly lower than those expected to give an analgesic or an anti-inflammatory effect by an action on this receptor.

Alternatively, appropriate therapeutic concentrations of the compound are expected to be approximately 10-20 times the Ki for an A_{2A} adenosine receptor at pH 5.5. Thus, for spongosine 15 to 30 nM is required whereas using the Ki at pH7.4 the concentration that is expected to be required is 20 to 30

It is expected that the amount of the compound that is administered should be 0.001-15 mg/kg. The amount may be less than 6 mg/kg. The amount may be at least 0.001, 0.01, 0.1, or 0.2 mg/kg. The amount may be less than 0.1, or 0.01 mg/kg. Preferred ranges are 0.001-10, 0.001-5, 0.001-2, 0.001-1, 0.001-0.1, 0.001-0.01, 0.01-15, 0.01-10, 0.01-5, 0.01-2, 0.01-1, 0.1-10, 0.1-5, 0.1-2, 0.1-1, 0.1-0.5, 0.1-0.4, 0.2-15, 0.2-10, 0.2-5, 0.2-2, 0.2-1.2, 0.2-1, 0.6-1.2, mg/kg.

Preferred doses for a human subject (for example a 70 kg subject) are less than 420 mg, preferably less than 28 mg, more preferably less than 21 mg, and preferably at least 0.07, 0.1, 0.7, or 0.8 mg, more preferably at least 3.5 or 7 mg. More preferably 7-70 mg, 14-70 mg, or 3.5-21 mg.

It is believed that the dosage amounts specified above are significantly lower (up to approximately 1000 times lower) than would be expected to be required for an analgesic or an anti-inflammatory effect based on the EC50 value of the compound at the adenosine A2A receptor.

The preferred dosage amounts specified above are aimed at producing plasma concentrations that are approximately one hundredth to one half of the EC50 value of the compound at an A_{2A} adenosine receptor.

It will be appreciated that an appropriate dosage of the A_{2A} adenosine receptor agonist may be less than the dosage that would be required in the absence of the calcium channel blocker because of the enhanced effect of the A_{2A} adenosine receptor agonist in the presence of the calcium channel blocker. A lower dose of the A_{2A} adenosine receptor agonist may be particularly advantageous, for example if any side effects of the agonist are reduced at the lower dose.

The combined preparation of the invention may be administered with or without other therapeutic agents, for example analgesics or anti-inflammatories (such as opiates, steroids, NSAIDs, cannabinoids, tachykinin modulators, or bradykinin modulators) or anti-hyperalgesics (such as gabapentin, pregabalin, cannabinoids, sodium or calcium channel modulators, anti-epileptics or anti-depressants), or DMARDs.

The exact regimen for administration of the combined preparation disclosed herein will necessarily be dependent upon the needs of the individual subject being treated, the type of treatment and, of course, the judgment of the attending practitioner.

A suitable dose of the calcium channel blocker will preferably be a dose of the compound conventionally used to decrease blood pressure in individuals with hypertension. Such doses will be known to the skilled person. For example, Amlodipine may be administered to a human subject at 2.5-10 mg per day, Benidipine at 2-4 mg once per day, Cilnidipine at 5-10 mg once per day, Clevidipine at 1-32 mg per hour (IV infusion) not exceeding an average of 21 mg per hour per 24 hours, Lacidipine at 2-6 mg once per day, Nimodipine at 60 mg every 4 hours (oral) or 1-2 mg per hour (IV infusion).

Embodiments of the invention are now described by way of example only, with reference to the accompanying drawings in which:

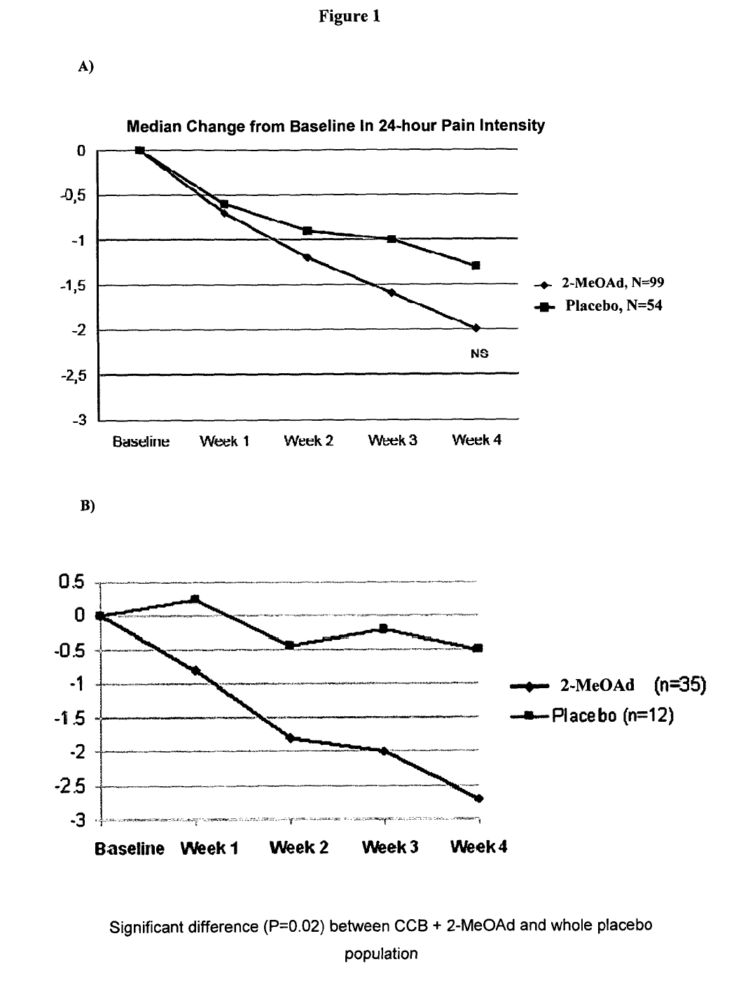

FIG. 1 shows the results from a Phase 2A clinical trial in which patients suffering from diabetic neuropathy were asked to rate their pain intensity once per week following administration of 2-methoxyadenosine (2-MeOAd) or a placebo. FIG. 1A shows the median change from baseline in 24-hour pain intensity over 4 weeks for the whole patient population. FIG. 1B shows the median change for a sub-group of the patient population who were administered with a calcium channel blocker as well as 2-methoxyadenosine or a placebo.

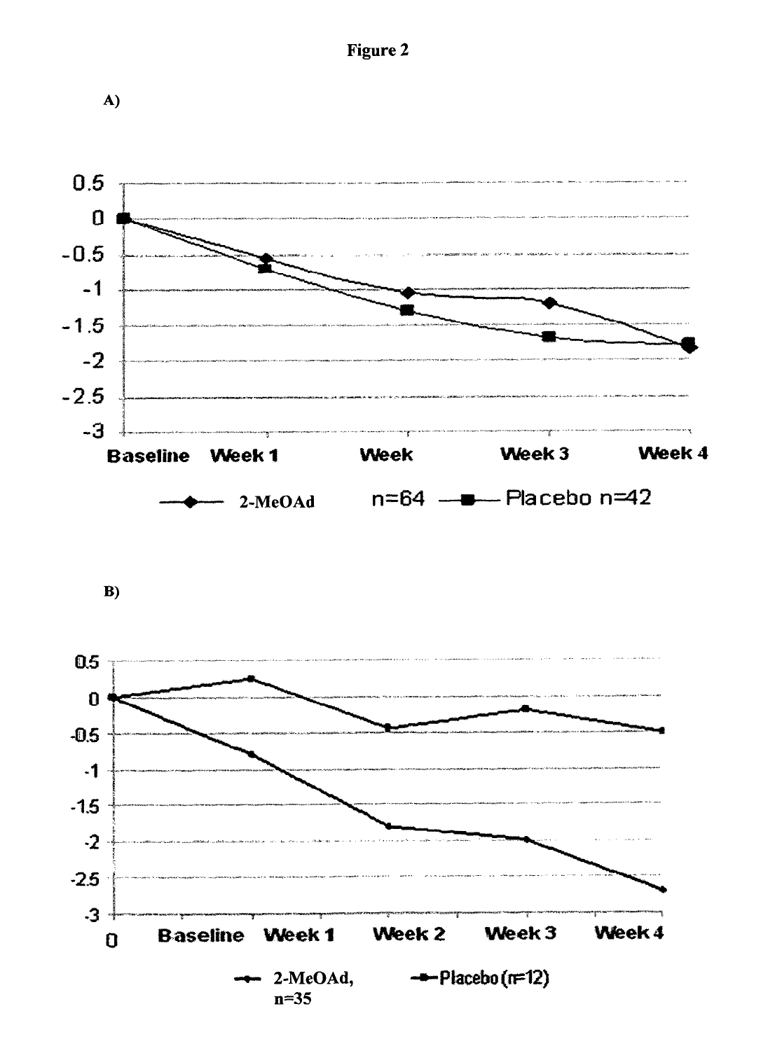

FIG. 2A shows the results from the Phase 2A clinical trial for patients who were not being administered a calcium channel blocker, and FIG. 2B shows the results for patients who were being administered a calcium channel blocker; and

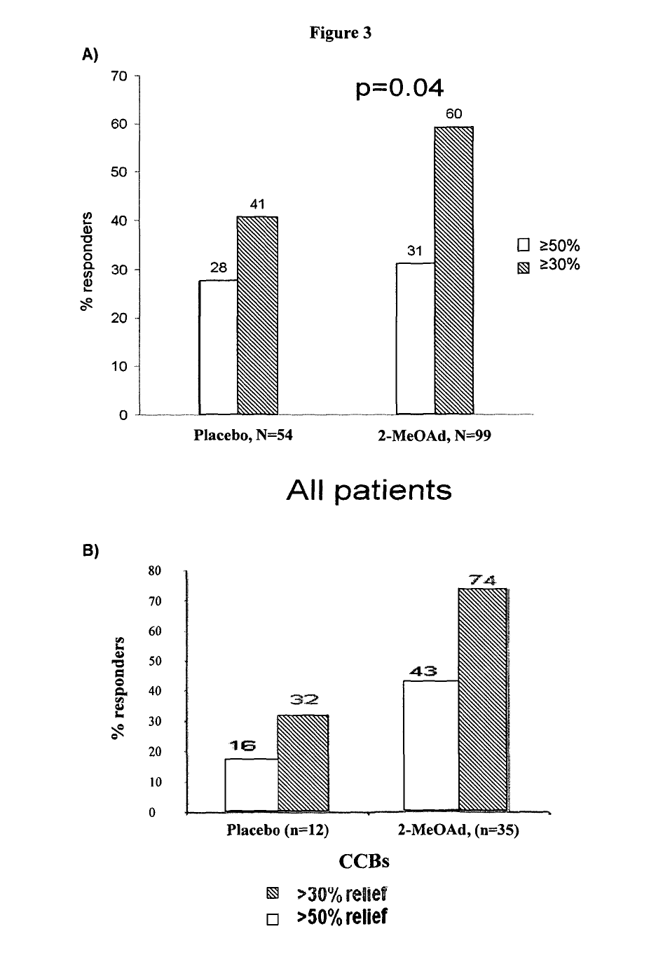

FIG. 3 shows the responder rate for patients in the Phase 2A clinical trial for patients administered with 2-methoxyadenosine (2-MeOAd)) or a placebo. FIG. 3A shows the results for all patients administered with 2-methoxyadenosine or a placebo, and FIG. 3B shows the results for patients being administered with a calcium channel blocker who were administered with 2-methoxyadenosine or a placebo. In each case, the darker shaded bar to the left represents patients with >50% relief, and the lighter shaded bar to the right represents patients with >30% relief.

### EXAMPLE

### Effect of 2-Methoxyadenosine on Diabetic Neuropathic Pain in Patients Also Taking Calcium Channel Blockers

In a Phase 2A clinical trial, patients suffering from diabetic neuropathy were administered 2-methoxyadenosine (2-MeOAd)) (7 mg 3 times per day) or a placebo over a four week period, and asked to rate their pain intensity (diabetic neuropathic pain) once per week based on an 11 point Likert Scale (0-10). The patients included some who were already being administered with a calcium channel blocker at a conventional therapeutic dose. The calcium channel blockers administered included Amlodipine (5-10 mg once per day), Verapamil, or Felodipine.

The median change from baseline in 24-hour pain intensity for the patient population (i.e. those being administered a calcium channel blocker and those not being administered a calcium channel blocker) is shown in FIG. 1A. The results show that by Week 4 the median change for patients administered 2-methoxyadenosine was 0.7 below those administered with the placebo. The results specifically for those patients who were administered with a calcium channel blocker as well as 2-methoxyadenosine or placebo are shown in FIG. 1B. The results show a significant difference (P=0.02) in median change between those patients already being administered with a calcium channel blocker who were administered with 2-methoxyadenosine compared to those given the placebo. For patients administered with 2-methoxyadenosine, the median change from baseline after 4 weeks was more than −2.5 (P<0.01), compared with −0.5 for those given the placebo.

FIG. 2 shows the median change from baseline in 24-hour pain intensity for patients administered with 2-methoxyadenosine (2-MeOAd) or a placebo. FIG. 2A shows the results for those patients who were not also administered with a calcium channel blocker, and FIG. 2B shows the results for those patients that were also administered with a calcium channel blocker. The results show that there was no effect of 2-methoxyadenosine in those patients who were not also administered with a calcium channel blocker, but a significant effect of 2-methoxyadenosine on pain intensity was observed compared with placebo in those patients who were also administered with a calcium channel blocker.

FIG. 3 shows the percentage of responders from those patients in the clinical trial. FIG. 3A shows the responder rate for all of the patients in the trial, and FIG. 3B shows the responder rate just for the patents administered 2-methoxyadenosine or placebo who were also administered with a calcium channel blocker. The results show that 74% patients who were also administered with a calcium channel blocker experienced >30% pain relief, and 43% experienced >50% pain relief, when administered 2-methoxyadenosine, compared with 32% who experienced >30% pain relief and 16% who experienced >50% pain relief when administered the placebo.

Patients in the clinical trial also completed the Short form McGill pain questionnaire (SF-MPQ) (Melzack, Pain, 1987, August; 30(2):191-7). The main component of the SF-MPQ consists of 15 descriptors (11 sensory; 4 affective) which are rated on an intensity scale as 032 none, 1=mild, 2=moderate or 3=severe. Three pain scores are derived from the sum of the intensity rank values of the words chosen for sensory, affective and total descriptors. The SF-MPQ also includes the Present Pain Intensity (PPI) index of the standard MPQ and a visual analogue scale (VAS).

Those patients who were administered with 2-methoxyadenosine and a calcium channel blocker experienced a reduction in total pain intensity score (P<0.03), a reduction in pain rated by the VAS (P<0.02), and a reduction in present pain intensity (P<0.04).

There was also a reduction in the Global Impression of Change: Clinical global impression of change (CGIC)—7 point scale (P<0.02), and patient global impression of change (PGIC)—7 point scale (favourable effect).

1-20. (canceled)

21. A pharmaceutical composition comprising: an A_{2A} adenosine receptor agonist, a calcium channel blocker and a pharmaceutically acceptable carrier, excipient, or diluent.

22. The pharmaceutical composition of claim 21 wherein the A_{2A} adenosine receptor agonist comprises a compound of formula (I): 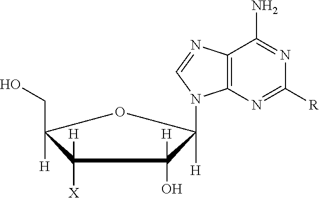
wherein R is C₁₋₄ alkoxy and X is OH or H;
or a pharmaceutically acceptable salt thereof.

23. The pharmaceutical composition of claim 21 wherein the A_{2A} adenosine receptor agonist comprises spongosine.

24. The pharmaceutical composition of claim 21 wherein A_{2A} adenosine receptor agonist comprises a compound of any of formula (II)-(VII): 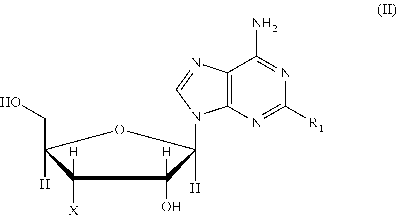
wherein:
when X═OH, R₁ is C₁ or C₄-C₆ alkoxy, OCH₂Cyclopropyl, OCH₂Cyclopentyl, O-(2,2,3,3-tetrafluoro-cycloButyl), phenoxy, substituted phenoxy, 4-methyl, phenyl (preferably 3-phenyl), 3-bromo, 3-isopropyl, 2-methyl, 2,4-difluoro, 2,5-difluoro, 3,4-difluoro, 2,3,5-trifluoro, or (3-methyl,4-fluoro)), OCH₂CH₂OH, OCH₂CHF₂, (5-indanyl)oxy, C₁, C₂, C₅, or C₆ alkylamino, (R) or (S)-sec-Butylamino, C₅ or C₆ cycloalkylamino, exo-norbornane amino, (N-methyl, N-isoamylamino), phenylamino, phenylamino with either methoxy or fluoro substituents, a C₂ sulfone group, a C₇ alkyl group, a cyano group, a CONH₂ group, or 3,5-dimethylphenyl; or
when X═H, R₁ is n-hexyloxy; 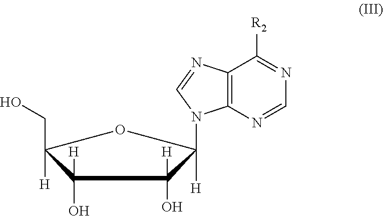
wherein R₂ is NMe₂, N-(2-isopentenyl), piperazinyl, (N-Me, N-benzyl), (N-Me, N—CH₂Ph(3-Br)), (N-Me, N—CH₂Ph(3-CF₃)), or (N-Me, N-(2-methoxyethyl)), or OCH₂Cyclopentyl; 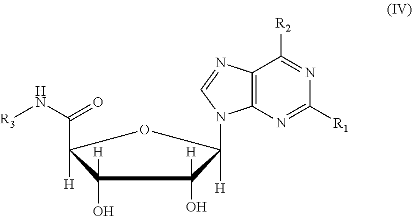
wherein:
when R₁═H, R₃ is an isopropyl group, and R₂ is either NH₂, a methylamino group (NHMe) or an isoamyl group (CH₂CH₂CHMe₂); or
when R₁═H, R₃ is H, and R₂ is NH₂; or
when R₁ is OMe, R₃ is Ph, and R₂ is NH₂; or
when R₁ is NHCH₂CH₂CH₂CH₂CH₂Me, R₃ is CH₂CH₂CH₂Me, and R₂ is NH₂; 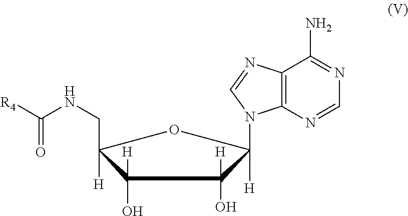
wherein R₄ is n-propyl or NHCH₂CH₃; 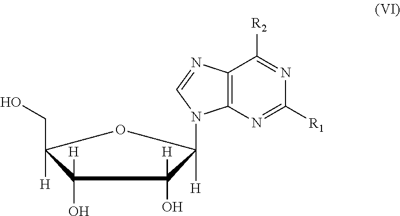
wherein:
R₁ is NHCyclohexyl when R₂ is NMe₂; or
R₁ is OMe when R₂ is NHBenzyl; 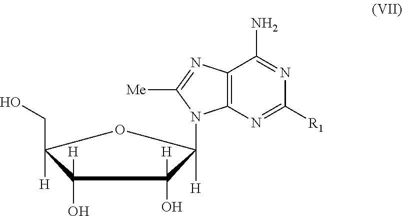
wherein R1 is NHCyclohexyl, NHCyclopentyl, or NH-n-Hexyl;
or a pharmaceutically acceptable salt thereof.

25. The pharmaceutical composition of claim 21 wherein the calcium channel blocker comprises a dihydropyridine, a phenylalkylamine, or a benzothiazepine.

26. A method for treating pain, inflammation, cancer, auto-immune disease, ischemia-reperfusion injury, epilepsy, sepsis, septic shock, or neurodegeneration, comprising administering the pharmaceutical composition of claim 21 to a subject in need thereof.

27. A method treating a vascular complication of diabetes comprising administering the pharmaceutical composition of claim 21 to a subject in need thereof.

28. The method of claim 27 wherein the vascular complication is a microvascular complication.

29. The method of claim 28 wherein the microvascular complication is selected from diabetic neuropathy, diabetic neuropathic pain, diabetic skin ulceration and dermopathy, diabetic kidney disease, or diabetic retinopathy.

30. The method of claim 27 wherein the vascular complication is or macrovascular complication.

31. The method of claim 30 wherein the macrovascular complication is cardiovascular disease or heart disease.

32. The method of claim 26 or claim 27 wherein the A_{2A} adenosine receptor agonist and the calcium channel blocker are co-administered to the subject.

33. The method of claim 26 or claim 27 wherein the A_{2A} adenosine receptor agonist and the calcium channel blocker are administered sequentially to the subject.

34. The method of claim 26 or claim 27 according to claim 21, wherein the A_{2A} adenosine receptor agonist and the calcium channel blocker are administered to the subject within 24 hours of each other.
